(19) United States
(12) Patent Application Publication
(10) Pub. No.: US 2026/0265787 A1
(43) Pub. Date: Sep. 10, 2026

(54) REDUCED FERMENTATION GLYCEROL AND ACETATE USING YEAST EXPRESSING RUBISCO FROM GALLIONELLA CAPSIFERRIFORMANS

(71) Applicant: Danisco US Inc., Palo Alto, CA (US)

(72) Inventor: Joseph F. Tuminello, Wilmington, DE (US)

(21) Appl. No.: 19/168,748

(22) PCT Filed: Mar. 26, 2024
(86) PCT No.: PCT/US2024/021531

§ 371 Date: Sep. 24, 2025

Related U.S. Application Data
(60) Provisional application No. 63/492,252, filed on Mar. 27, 2023.

(51) Int. Cl. C12P 7/06; C07K 14/245; C12N 1/18; C12N 9/04; C12N 9/12; C12N 9/88; C12R 1/85

(52) U.S. Cl. CPC C12P 7/06; C07K 14/245; C12N 1/18; C12N 9/0006; C12N 9/1205; C12N 9/88; C12R 2001/85; C12Y 101/01006; C12Y 207/01019; C12Y 207/01029; C12Y 401/01039

(57) ABSTRACT

The present compositions and methods relate to modified yeast cells that heterologously express the RuBisCo enzyme from a particular species of iron-oxidizing bacteria. The modified yeast cells demonstrate reduced glycerol and acetate accumulation in fermentation, while maintaining high ethanol production, making them useful for large-scale ethanol production from starch substrates, where glycerol and acetate represent undesirable by-products.

### CROSS-REFERENCE TO RELATED APPLICATION

This application claims priority from U.S. Provisional Application No. 63/492,252, filed Mar. 27, 2023, the contents of which are hereby incorporated by reference in their entirety.

### INCORPORATION BY REFERENCE OF THE SEQUENCE LISTING

The present application is being filed with a Sequence Listing in electronic format. The Sequence Listing is provided as a file entitled NB42202WOPCTSeqList.xml, created on Mar. 7, 2024, which is 25,628 bytes in size. The information in the electronic format of the Sequence Listing is incorporated by reference in its entirety.

### TECHNICAL FIELD

The present compositions and methods relate to modified yeast cells that heterologously express the RuBisCo enzyme from a particular species of iron-oxidizing bacteria. The modified yeast cells demonstrate reduced glycerol and acetate accumulation in fermentation, while maintaining high ethanol production, making them useful for large-scale ethanol production from starch substrates, where glycerol and acetate represent undesirable by-products.

### BACKGROUND

First-generation yeast-based ethanol production converts sugars into fuel ethanol. The annual fuel ethanol production by yeast is about 90 billion liters worldwide (Gombert, A. K. and van Maris. A. J. (2015) Curr. Opin. Biotechnol. 33:81-86). It is estimated that about 70% of the cost of ethanol production is the feedstock. These numbers have remained largely unchanged for roughly a decade. Since the production volume is so large, even small improvements in yield have massive economic impact across the industry.

Ribulose-1,5-bisphosphate carboxylase-oxygenase, commonly known as RuBisCo, is an enzyme involved in the first major step of carbon fixation, in which atmospheric carbon dioxide is converted by plants and other photosynthetic organisms to energy-rich molecules, such as glucose. The enzyme catalyzes the carboxylation of ribulose-1,5-bisphosphate (also known as RuBP) and is a key component of the mechanism by which autotrophic bacteria, algae, and terrestrial plants fix CO₂ into organic biomass via the Calvin-Benson-Basham reductive pentose phosphate pathway.

Heterologous expression of a proteobacterial RuBisCo from Thiobacillus dentrificans, and bacterial protein chaperones in Saccharomyces cerevisiae has been described (see, generally, WO2014129898, WO2017216136, WO2018114762, WO2019063542, WO2019063543 and WO2020043497). Notwithstanding, the need exists to improve ethanol production efficiency and reduce the production of undesirable side products, particularly engineered yeast that tend to produce an increased amount of acetate.

### SUMMARY

The present compositions and methods relate to modified yeast cells that heterologously express RuBisCo from the iron-oxidizing chemolithotrophic bacterium Gallionella capsiferriformans. The modified yeast cells demonstrate reduced glycerol and acetate accumulation in fermentation using a starch substrate. Aspects and embodiments of the modified yeast cells and methods are described in the following, independently numbered paragraphs.
1. In one aspect, modified yeast cells derived from parental yeast cells are provided, the modified cells comprising a genetic alteration that causes the modified cells to produce the ribulose-1,5-bisphosphate carboxylase-oxygenase (RuBisCo) enzyme from Gallionella capsiferriformans (GcRuBisCo), wherein the modified cells produce during fermentation a decreased amount of glycerol and/or acetate compared to the amount of glycerol and acetate produced by otherwise identical parental cells producing the RuBisCo enzyme from Thiobacillus dentrificans under identical fermentation conditions, and wherein the modified yeast cells and parental yeast cells both further comprise an exogenous gene encoding a phosphoribulokinase.
2. In some embodiments of the modified cells of paragraph 1, the genetic alteration comprises the introduction into the parental cells of a nucleic acid capable of directing the expression of GcRuBisCo.
3. In some embodiments of the modified cells of paragraph 2, the genetic alteration comprises the introduction of an expression cassette for directing the expression of the GcRuBisCo.
4. In some embodiments, the modified cells of any of paragraphs 1-3 further comprise exogenous genes encoding chaperone proteins.
5. In some embodiments of the modified cells of paragraph 4, the chaperone proteins are GroES and GroEL from Escherichia coli
6. In some embodiments, the modified cells further comprise an exogenous gene encoding a glycerol transporter, a glycerol dehydrogenase and dihydroxyacetone kinase.
7. In some embodiments of the modified cells of any of paragraphs 1-6, the cells further comprise one or more genes of the phosphoketolase pathway.
8. In some embodiments, the modified cells of any of paragraphs 1-7 further comprise an alteration in the glycerol pathway and/or the acetyl-CoA pathway.
9. In some embodiments, the modified cells of any of paragraphs 1-8 further comprise an alternative pathway for making ethanol.
10. In some embodiments, the modified cells of any of paragraphs 1-9 further comprise an exogenous gene encoding a molecular chaperone.
11. In some embodiments of the modified cells of any of paragraphs 1-10, the cells further comprise an exogenous gene encoding a carbohydrate processing enzyme.
12. In some embodiments of the modified cells of any of paragraphs 1-11, the cells are of a Saccharomyces spp.
13. In another aspect, a method for decreasing the production of glycerol and acetate from yeast cells grown on a carbohydrate substrate is provided, comprising: introducing into parental yeast cells a genetic alteration that causes the modified cells to produce ribulose-1,5-bisphosphate carboxylase-oxygenase (RuBisCo) from Gallionella capsiferriformans.
14. In some embodiments of the method of paragraph 13, the modified cells are the cells of any of paragraphs 1-12.

These and other aspects and embodiments of modified cells and methods will be apparent from the description, including any accompanying Drawings/FIGURES. Each of the aspects and embodiments described herein are capable of being used together, unless excluded either explicitly or clearly from the context of the embodiment or aspect.

### DETAILED DESCRIPTION

### I. Definitions

Prior to describing the modified yeast cells and methods in detail, the following terms are defined for clarity. Terms not defined should be accorded their ordinary meanings as used in the relevant art.

Definitions of terms may appear throughout the specification. It is to be understood that this disclosure is not limited to particular embodiments described, as such may, of course, vary. It is also to be understood that the terminology used herein is for the purpose of describing particular embodiments only and is not intended to be limiting.

It must be noted that as used herein and in the appended claims, the singular forms “a,” “an,” and “the” include plural referents unless the context clearly dictates otherwise. For example, “a” or “an” include “at least one” and “one or more.”

The terms “comprising”, “comprises,” and “comprised of” as used herein are synonymous with “including,” “includes,” “containing,” “contains,” “has,” and “having,” and grammatical variants thereof, and are inclusive or open-ended and do not exclude additional, non-recited members, elements, or method steps. The terms “comprising,” “comprises,” “comprised of,” “including,” “includes,” “containing,” “contains,” “has,” “having” and grammatical variants thereof also include the term “consisting of.”

As used herein, the term “alcohol” refers to an organic compound in which a hydroxyl functional group (—OH) is bound to a saturated carbon atom.

As used herein, the terms “yeast cells,” “yeast strains,” or simply “yeast” refer to organisms from the phyla Ascomycota and Basidiomycota. Exemplary yeast is budding yeast from the order Saccharomycesles. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “engineered yeast cells,” “variant yeast cells,” “modified yeast cells,” or similar phrases, refer to yeast that include genetic modifications and characteristics described herein. Variant/modified yeast do not include naturally occurring yeast.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequences are presented from an N-terminal to C-terminal direction. The polymer can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids etc.), as well as other modifications known in the art.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype. The term “allele” is generally preferred when an organism contains more than one similar gene, in which case each different similar gene is referred to as a distinct “allele.”

As used herein, the term “expressing a polypeptide” and similar terms refers to the cellular process of producing a polypeptide using the translation machinery (e.g., ribosomes) of the cell.

As used herein, an “expression cassette” refers to a DNA fragment that includes a promoter, and amino acid coding region and a terminator (i.e., promoter::amino acid coding region::terminator) and other nucleic acid sequence needed to allow the encoded polypeptide to be produced in a cell. Expression cassettes can be exogenous (i.e., introduced into a cell) or endogenous (i.e., extant in a cell).

As used herein, the terms “wild-type” and “native” are used interchangeably and refer to genes, proteins or strains found in nature, or that are not intentionally modified for the advantage of the presently described yeast.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed. The protein of interest is encoded by an endogenous gene or a heterologous gene (i.e., gene of interest”) relative to the parental strain. The protein of interest can be expressed intracellularly or as a secreted protein.

The term “heterologous” when used in reference to a polynucleotide, a gene, a polypeptide, or an enzyme refers to a polynucleotide, gene, polypeptide, or an enzyme not normally found in the host organism. “Heterologous” also includes a native coding region, or portion thereof, that is reintroduced into the source organism in a form that is different from the corresponding native gene, e.g., not in its natural location in the organism's genome. The heterologous polynucleotide or gene may be introduced into the host organism by, e.g., gene transfer. A heterologous gene may include a native coding region that is a portion of a chimeric gene including non-native regulatory regions that is reintroduced into the native host. Foreign genes can comprise native genes inserted into a non-native organism, or chimeric genes.

As used herein, the terms “genetic manipulation,” “genetic alteration,” “genetic engineering,” and similar terms are used interchangeably and refer to the alteration/change of a nucleic acid sequence. The alteration can include but is not limited to a substitution, deletion, insertion or chemical modification of at least one nucleic acid in the nucleic acid sequence.

As used herein, “aerobic fermentation” refers to growth and production process in the presence of oxygen.

As used herein, “anaerobic fermentation” refers to growth and production in the absence of oxygen.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:
following meanings unless otherwise specified:
   ° C. degrees Centigrade
   bp base pairs
   CO₂ carbon dioxide
   DNA deoxyribonucleic acid
   ds or DS dry solids
   EC enzyme commission
   EtOH ethanol
   g or gm gram
   GcRuBisCo RuBisCo from Gallionella capsiferriformans
   g/L grams per liter
   H₂O water
   HPLC high performance liquid chromatography
   hr or h hour
   kg kilogram
   M molar
   mg milligram
   min minute
   mL or ml milliliter
   mM millimolar
   N normal
   nm nanometer
   PCR polymerase chain reaction
   ppm parts per million
   RuBisCo ribulose-1,5-bisphosphate carboxylase-oxygenase
   RuBP ribulose-1,5-bisphosphate
   Δ relating to a deletion
   μg microgram
   μL and μl microliter
   μM micromolar

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Numerical values and ranges may be presented herein with the numerical value being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context. All values and ranges implicitly include the term “about” unless the context clearly dictates otherwise.

All publications, including patent documents, scientific articles, and databases, referred to in this application are incorporated by reference in their entirety for all purposes to the same extent as if each individual publication were individually incorporated by reference. Nothing herein is to be construed as an admission that such publications constitute prior art to the claims appended hereto. If a definition set forth herein is contrary to or otherwise inconsistent with a definition set forth in the patents, applications, published applications, and other publications that are herein incorporated by reference, the definition set forth herein prevails over the definition that is incorporated herein by reference.

### II. Modified Yeast Cells Expressing RuBisCo from G. Capsiferrifirmans

Ribulose-1,5-bisphosphate carboxylase-oxygenase (RuBisCo; EC 4.1.1.39) catalyzes the carboxylation of ribulose-1,5-bisphosphate (RuBP). RuBisCo is involved with the first major step of carbon fixation, by which atmospheric carbon dioxide is converted by plants and other photosynthetic organisms to energy-rich molecules, such as glucose.

The present modified yeast cells and methods are based on the unexpected observation that the RuBisCo from a particular Gallionella sp., namely Gallionella capsiferrifirmans, when heterologously expressed in high ethanol-producing parental yeast cells, reduces glycerol and acetate accumulation following fermentation, compared to the amounts obtained with a corresponding parental strain that instead produces the RuBisCo from Thiobacillus dentrificans.

In some embodiments, the RuBisCo from G. capsiferriformans, (herein, “GcRuBisCo”) is expressed (or produced) in the modified cells by introducing into a parental yeast cell a nucleic acid capable of directing the expression of GcRuBisCo. In some embodiments, the GcRuBisCo has the sequence set forth by SEQ ID NO: 1. Particular methods include but are not limited to (i) introducing additional copies of an endogenous expression cassette for increased production of the polypeptide into a host cell, (ii) introducing an exogenous expression cassette(s) for increased production of polypeptide into a host cell, (iii) substituting an endogenous cassette with an exogenous expression cassette that allows the production of an increased amount of the polypeptide, (iv) modifying or replacing the promoter of an endogenous expression cassette to increase expression, and/or (v) modifying any aspect of the host cell to increase the half-life of the polypeptide in the host cell. In some embodiments, an exogenous expression cassette is introduced into a parental yeast cell to produce GcRuBisCo in the yeast cell.

In some embodiments, a gene encoding a RuBisCo enzyme that is substantially similar to GcRuBisCo is expressed. Substantially similar RuBisCo enzyme are those that provide similar advantages to GcRuBisCo, compared to RuBisCo from Thiobacillus denitrificans In some embodiments, the amino acid sequence of the RuBisCo enzyme has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more compared to a polypeptide having the amino acid sequence of SEQ ID NO: 1. In some embodiments, the amino acid sequence of the RuBisCo enzyme has at least 60% identity compared to a polypeptide having the amino acid sequence of SEQ ID NO: 1. In some embodiments, the amino acid sequence of the RuBisCo enzyme has at least 70% identity compared to a polypeptide having the amino acid sequence of SEQ ID NO: 1. In some embodiments, the amino acid sequence of the RuBisCo enzyme has at least 75% identity compared to a polypeptide having the amino acid sequence of SEQ ID NO: 1. In some embodiments, the amino acid sequence of the RuBisCo enzyme has at least 80% identity compared to a polypeptide having the amino acid sequence of SEQ ID NO: 1. In some embodiments, the amino acid sequence of the RuBisCo enzyme has at least 85% identity compared to a polypeptide having the amino acid sequence of SEQ ID NO: 1. In some embodiments, the amino acid sequence of the RuBisCo enzyme has at least 90% identity compared to a polypeptide having the amino acid sequence of SEQ ID NO: 1. In some embodiments, the amino acid sequence of the RuBisCo enzyme has at least 95% identity compared to a polypeptide having the amino acid sequence of SEQ ID NO: 1. In some embodiments, the amino acid sequence of the RuBisCo enzyme has at least 96% identity compared to a polypeptide having the amino acid sequence of SEQ ID NO: 1. In some embodiments, the amino acid sequence of the RuBisCo enzyme has at least 97% identity compared to a polypeptide having the amino acid sequence of SEQ ID NO: 1. In some embodiments, the amino acid sequence of the RuBisCo enzyme has at least 98% identity compared to a polypeptide having the amino acid sequence of SEQ ID NO: 1. In some embodiments, the amino acid sequence of the RuBisCo enzyme has at least 99% identity compared to a polypeptide having the amino acid sequence of SEQ ID NO: 1. In particular embodiment, the amino acid sequence of the RuBisCo enzyme has at least 80%, at least 85%, at least 90%, at least 91%, at least 92%, at least 93%, at least 94%, at least 95%, at least 96%, at least 97%, at least 98%, at least 99% or more, compared to a polypeptide having the amino acid of SEQ ID NO: 1. In some embodiments, the amino acid sequence of the RuBisCo enzyme has the amino acid sequence of SEQ ID NO: 1.

The amino acid sequence of GcRuBisCo is
shown, below, as SEQ ID NO: 1:
MDQSNRYANLNLKEEDLIKNGKHLLVAYKLIPAKGHGFLEVAAHVAAESS
TGINVEVSTTDDFTRGVDALVYEIDETAFGDDIVKGGGLFKVAYPVELFD
PNLTDGTYNISHMWSLILGNNQGMGDHQGLRMLDFLVPEMMVRKFDGPSA
NISNLWKVLGRSETDGGYIAGTIIKPKLGLRPEPFAKACYDFWLGGDFIK
NDEPQANQPFCPMEVVMPKVAEAMDRAQQETGQAKLFSANITADYYKEMI
HRGDEVLETFAKYNSASHVAFLVDGFVTGPAGVTTCRREFPDTFLHFHRA
GHGAVTSYKSPMGMDPLCYMKLVRLMGASGMHTGTMGYGKMEGHGKETVL
AYMLERDECQGPYFYQKWYGMKATTPIISGGMNALRLPGFFQNLGHGNVI
NTCGGGAFGHIDSPAAGGISLGQAYDCWKSGSDPIEYAKTHKEFARAFES
FPKDGDKLFAGWREKLGVHK

In some embodiments, the nucleic acid sequence of the RuBisCo has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more identity, compared to the nucleic acid sequence of SEQ ID NO: 2. In some embodiments, the nucleic acid sequence of the GcRuBisCo is the nucleic acid sequence of SEQ ID NO: 2.

Preferably, expressing GcRuBisCo in yeast cells is achieved by genetic manipulation using sequence-specific molecular biology techniques, as opposed to chemical mutagenesis, which is generally not targeted to specific nucleic acid sequences. Nonetheless, chemical mutagenesis is not excluded as a method for making the present modified yeast cells. In some embodiments, genetic manipulation includes using an established gene editing technique, e.g., CRISPR/Cas editing, zing finger endonucleases (ZFN), transcriptional activator like endonucleases (TALENs). In some embodiments, CRISPR/Cas9 editing is used. Compositions and methods for fungal genome modification by CRISPR/Cas9 systems are described and well known in the art (e.g., see, PCT Publication Nos: WO2016/100571, WO2016/100568, WO2016/100272, WO2016/100562 and the like).

In some embodiments the parental yeast cells are otherwise genotypically identical to the modified yeast cells. In some embodiments the parental yeast cells are otherwise genotypically identical to the modified yeast cells with respect to metabolic pathways involved with ethanol production. In some embodiments, the parental yeast cells, before and after modification as described, herein, express RuBisCo from Thiobacillus denitrificans. In some embodiments, the parental yeast cells, prior to modification as described, herein, expressed RuBisCo from Thiobacillus denitrificans.

In some embodiments the parental yeast cells are already genetically modified, whether using standard molecular biology techniques, by chemical mutagenesis, by mating, by stress-selection and adaptation or by other methods, to improve ethanol production in moderate to high dissolved solids starch liquefact. Examples of such yeast cells are commercially available and include those marketed under the names SYNERXIA® (IFF, US), EBOOST® (DSM, NE), TRANSFERM® (Lallemand, CA) and INNOVA® (Novozymes, DK).

It will be understood that reference to a parental yeast cell or strain is made to describe the contribution of expressing GcRuBisCo to a desirable phenotype. Yeast selected for modification by expressing GcRuBisCo are likely to already be high ethanol producing yeast that include additional genetic modifications directed to achieving the same phenotype, and it may be difficult to determine the individual contribution of each genetic mutation.

In some embodiments the reduction of glycerol following fermentation by modified yeast cells expressing GcRuBisCo is a decrease of at least 1%, at least 2%, at least 3%, at least 4%, at least 5%, at least 6%, at least 7%, at least 8%, at least 9%, or even at least 10%, or more, compared to the amount of glycerol produced by parental yeast cells grown under the same conditions.

In some embodiments the reduction of acetate following fermentation by modified yeast cells expressing GcRuBisCo is a reduction of at least 5%, at least 10%, at least 15%, or even at least 20%, or more, compared to the amount of acetate produced by otherwise identical parental yeast cells grown under the same conditions.

In some embodiments, the decrease in acetate produced by the modified yeast cells is a decrease of at least 1%, at least 2%, at least 3%, at least 4%, at least 5%, or more, compared to the amount of acetate produced by parental yeast cells growing under the same conditions.

In some embodiments, the reduction in the amount of residual glucose following fermentation by modified yeast cells expressing GcRuBisCo is a reduction of at least 10%, at least 20%, at least 30%, at least 40%, at least 50%, or even at least 60%, or more, compared to the amount of residual glucose present following fermentation by an otherwise identical parental yeast grown under the same conditions.

In some embodiments, the amount of ethanol produced in yeast expressing GcRuBisCo is approximately the same, or even more, e.g., at least 0.5%, at least 1.0%, at least 2.0%, or even at least 30%, or more, compared to the amount of ethanol produced by the otherwise identical parental yeast grown under the same conditions. In some embodiments, the amount of ethanol produced in yeast expressing GcRuBisCo is approximately the same, or even more, e.g., at least 0.5%, at least 1.0%, at least 2.0%, or even at least 3.0%, or more, compared to the amount of ethanol produced by the otherwise identical parental yeast grown under the same conditions.

### III. Modified Yeast Cells with Additional RuBisCo-Associated Modifications

Yeast cells harboring genes encoding RuBisCo and phosphoribulokinase (EC 2.7.1.19) have been described (WO2014/129898). Introducing one or more genes encoding an NAD⁺-linked glycerol dehydrogenase (EC 1.1.1.6 or EC 1.1.1.72) and one or more genes encoding a dihydroxyacetone kinase (E.C. 2.7.1.28 or E.C. 2.7.1.29) appears to further increase ethanol production (WO2018114762A1).

In some embodiments, the present modified yeast cells, in addition to expressing GcRuBisCo, further include a gene for the heterologous expression of phosphoribulokinase and/or dihydroxyacetone kinase, optionally in combination with genes encoding a glycerol dehydrogenase. The yeast may further include a modification in a gene involved in glycerol synthesis, as described in more detail below. The yeast cells may optionally express a heterologous gene encoding a glycerol transporter, a glycerol exporter and/or a glycerol kinase.

In some embodiments, the yeast cells further include a gene encoding a molecular chaperone. Chaperones may originate from prokaryotes. In particular embodiments, the chaperone is Escherichia coli GroEL and/or GroES. In some embodiments, the amino acid sequence of the GroEL has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a polypeptide having the amino acid of SEQ ID NO: 3. In some embodiments, the amino acid sequence of the GroEL has at least 60% identity compared to a polypeptide having the amino acid of SEQ ID NO: 3. In some embodiments, the amino acid sequence of the GroEL has at least 65% identity compared to a polypeptide having the amino acid of SEQ ID NO: 3. In some embodiments, the amino acid sequence of the GroEL has at least 70% identity compared to a polypeptide having the amino acid of SEQ ID NO: 3. In some embodiments, the amino acid sequence of the GroEL has at least 75% identity compared to a polypeptide having the amino acid of SEQ ID NO: 3. In some embodiments, the amino acid sequence of the GroEL has at least 80% identity compared to a polypeptide having the amino acid of SEQ ID NO: 3. In some embodiments, the amino acid sequence of the GroEL has at least 85% identity compared to a polypeptide having the amino acid of SEQ ID NO: 3. In some embodiments, the amino acid sequence of the GroEL has at least 90% identity compared to a polypeptide having the amino acid of SEQ ID NO: 3. In some embodiments, the amino acid sequence of the GroEL has at least 95% identity compared to a polypeptide having the amino acid of SEQ ID NO: 3. In some embodiments, the amino acid sequence of the GroEL is the amino acid of SEQ ID NO: 3. In some embodiments, the nucleic acid sequence of the GroEL has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a the nucleic acid sequence of SEQ ID NO: 4. In some embodiments, the amino acid sequence of the GroES has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a polypeptide having the amino acid of SEQ ID NO: 5. In some embodiments, the amino acid sequence of the GroES has at least 60% identity compared to a polypeptide having the amino acid of SEQ ID NO: 5. In some embodiments, the amino acid sequence of the GroES has at least 65% identity compared to a polypeptide having the amino acid of SEQ ID NO: 5. In some embodiments, the amino acid sequence of the GroES has at least 70% identity compared to a polypeptide having the amino acid of SEQ ID NO: 5. In some embodiments, the amino acid sequence of the GroES has at least 75% identity compared to a polypeptide having the amino acid of SEQ ID NO: 5. In some embodiments, the amino acid sequence of the GroES has at least 80% identity compared to a polypeptide having the amino acid of SEQ ID NO: 5. In some embodiments, the amino acid sequence of the GroES has at least 85% identity compared to a polypeptide having the amino acid of SEQ ID NO: 5. In some embodiments, the amino acid sequence of the GroES has at least 90% identity compared to a polypeptide having the amino acid of SEQ ID NO: 5. In some embodiments, the amino acid sequence of the GroES has at least 95% identity compared to a polypeptide having the amino acid of SEQ ID NO: 5. In some embodiments, the amino acid sequence of the GroES is the amino acid of SEQ ID NO: 5. In some embodiments, the nucleic acid sequence of the GroES has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a the nucleic acid sequence of SEQ ID NO: 6.

### IV. Modified Yeast Cells with Genes of an Exogenous PKL Pathway

Expressing GcRuBisCo (optionally with associated genes) can be combined with expression of genes in the PKL pathway to reduce the production of elevated amounts of acetate that is associated with introducing an exogenous PKL pathway into yeast.

Engineered yeast cells having a heterologous PKL pathway have been previously described (WO2015148272). These cells express heterologous phosphoketolase (PKL), phosphotransacetylase (PTA) and acetylating acetyl dehydrogenase (AADH), optionally with other enzymes, to channel carbon flux away from the glycerol pathway and toward the synthesis of acetyl-CoA, which is then converted to ethanol. Such modified cells are capable of increased ethanol production in a fermentation process when compared to otherwise-identical parent yeast cells.

### V. Modified Yeast Cells with Other Mutations that Affect Alcohol Production

In some embodiments, in addition to expressing GcRuBisCo, the present modified yeast cells may further include mutations that result in attenuation of the native glycerol biosynthesis pathway and/or reuse glycerol pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), 8,795,998 (Pronk et al.) and 8,956,851 (Argyros et al.). Methods to enhance the reuse glycerol pathway by over expression of glycerol dehydrogenase (GCY1) and dihydroxyacetone kinase (DAK1) to convert glycerol to dihydroxyacetone phosphate (Zhang et al. (2013) J. Ind. Microbiol. Biotechnol. 40:1153-60). In some embodiments, the DAK1 is a heterologous DAK1. In some embodiments, the DAK1 is an exogenous DAK1. In some embodiments, the amino acid sequence of the DAK1 has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a polypeptide having the amino acid sequence of SEQ ID NO: 11. In some embodiments, the amino acid sequence of the DAK1 has at least 60% identity compared to a polypeptide having the amino acid sequence of SEQ ID NO: 11. In some embodiments, the amino acid sequence of the DAK1 has at least 65% identity compared to a polypeptide having the amino acid sequence of SEQ ID NO: 11. In some embodiments, the amino acid sequence of the DAK1 has at least 70% identity compared to a polypeptide having the amino acid sequence of SEQ ID NO: 11. In some embodiments, the amino acid sequence of the DAK1 has at least 75% identity compared to a polypeptide having the amino acid sequence of SEQ ID NO: 11. In some embodiments, the amino acid sequence of the DAK1 has at least 80% identity compared to a polypeptide having the amino acid sequence of SEQ ID NO: 11. In some embodiments, the amino acid sequence of the DAK1 has at least 85% identity compared to a polypeptide having the amino acid sequence of SEQ ID NO: 11. In some embodiments, the amino acid sequence of the DAK1 has at least 90% identity compared to a polypeptide having the amino acid sequence of SEQ ID NO: 11. In some embodiments, the amino acid sequence of the DAK1 has at least 95% identity compared to a polypeptide having the amino acid sequence of SEQ ID NO: 11. In some embodiments, the amino acid sequence of the DAK1 is the amino acid sequence of SEQ ID NO: 11. In some embodiments, the nucleic acid sequence of the DAK1 has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to the nucleic acid sequence of SEQ ID NO: 12.

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-COA. This partially reduces the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present modified yeast cells and methods the yeast expressly lacks a heterologous gene(s) encoding an acetylating acetaldehyde dehydrogenase, a pyruvate-formate lyase or both.

In some embodiments, the present modified yeast cells may further over-express a sugar transporter-like (STL1) polypeptide to increase the uptake of glycerol (see, e.g., Ferreira et al. (2005) Mol. Biol. Cell. 16:2068-76; Dušková et al. (2015) Mol. Microbiol. 97:541-59 and WO2015023989 A1) to increase ethanol production and reduce acetate. In some embodiments, the STL1 is a heterologous STL1. In some embodiments, the STL1 is an exogenous STL1. In some embodiments, the amino acid sequence of the STL1 has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a polypeptide having the amino acid sequence of SEQ ID NO: 13. In some embodiments, the STL1 is an exogenous STL1. In some embodiments, the amino acid sequence of the STL1 has the amino acid sequence of SEQ ID NO: 13. In some embodiments, the nucleic acid sequence of the STL1 has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a the nucleic acid sequence of SEQ ID NO: 14.

In some embodiments, the present modified yeast cells may further over-express a glycerol dehydrogenase (GldA). In some embodiments, the GldA is a heterologous GldA. In some embodiments, the GldA is an exogenous GldA. In some embodiments the amino acid sequence of the GldA has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a polypeptide having the amino acid sequence of SEQ ID NO: 7. In some embodiments the amino acid sequence of the GldA has the amino acid sequence of SEQ ID NO: 7. In some embodiments, the nucleic acid sequence of the GldA has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a the nucleic acid sequence of SEQ ID NO: 8.

In some embodiments, the present modified yeast cells may further over-express a phosphoribulokinase (PRK). In some embodiments, the PRK is a heterologous PRK. In some embodiments, the PRK is an exogenous PRK. In some embodiments the amino acid sequence of the PRK has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a polypeptide having the amino acid sequence of SEQ ID NO: 9. In some embodiments the amino acid sequence of the PRK has the amino acid sequence of SEQ ID NO: 9. In some embodiments, the nucleic acid sequence of the PRK has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity or more, compared to a the nucleic acid sequence of SEQ ID NO: 10.

In some embodiments, the yeast cells further comprise a deletion, mutation, over-expression, and/or substitution in one or more endogenous polynucleotides encoding FRA2, ALD6, ADH1, GPD2, BDH1, DLS1, DPB3, CPR1, MAL23C, MNN4, PAB1, TMN2, HAC1, PTC1, PTC2, OSM1, GIS1, CRZ1, HUG1, GDS1, CYB2P, SFC1, MVB12, LDB10, C5SD, GIC1, GIC2, YMR226C, PHO13, ADH5 MIG1, MIG2, MIG3, JID1, KGD2, ARG7, LEU4, MET2, DAL7, ISN1, RSF2 and TDA9. In some embodiments, the yeast cells, e.g., modified yeast cells, further comprise a deletion, mutation, over-expression, and/or substitution in one or more endogenous polynucleotides encoding DLS1, CPR1, and PAB1. In some embodiments, the yeast cells, e.g., modified yeast cells, further over-express PAB1. In some embodiments, the yeast cells, e.g., modified yeast cells, further express a decreased amount or do not express DLS1. In some embodiments, the yeast cells, e.g., modified yeast cells, further include a deletion of a YJL065c gene. In some embodiments, the yeast cells, e.g., modified yeast cells, further express a decreased amount or do not express CPR1. In some embodiments, the yeast cells, e.g., modified yeast cells, further include a deletion of a YDR155c gene.

### VI. Modified Yeast Cells with Other Beneficial Mutations

In some embodiments, in addition to expressing GcRuBisCo, and optionally harboring other mutations that benefit alcohol production, the present modified yeast cells further include any number of additional genes of interest encoding proteins of interest. Additional genes of interest may be introduced before, during, or after genetic manipulations that result in expression GcRuBisCo. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transaldolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VII. Use of the Modified Yeast for Improved Alcohol Production

The present modified yeast cells and methods include methods for improving alcohol production and/or reducing glycerol production, in fermentation reactions. In some embodiments, the present modified yeast cells and methods include methods for increasing alcohol production and reducing glycerol production, in fermentation reactions. In some embodiments, the present modified yeast cells and methods include methods for reducing glycerol production in fermentation reactions. In some embodiments, the present modified yeast cells and methods include methods for increasing alcohol production and/or reducing acetate production, in fermentation reactions. In some embodiments, the present modified yeast cells and methods include methods for increasing alcohol production and reducing acetate production, in fermentation reactions. In some embodiments, the present modified yeast cells and methods include methods for reducing acetate production, in fermentation reactions. In some embodiments, the present modified yeast cells and methods include methods for increasing alcohol production and reducing acetate and glycerol production, in fermentation reactions. In some embodiments, the present modified yeast cells and methods include methods for reducing acetate and glycerol production, in fermentation reactions. In some embodiments, the present modified yeast cells and methods include methods for increasing the rate of alcohol production. In some embodiments, the increase in rate occurs early in fermentation reactions. Such methods are not limited to a particular fermentation process. The present engineered yeast is expected to be a “drop-in” replacement for convention yeast, or commercially available, high ethanol-producing yeast. While primarily intended for fuel alcohol production, the present yeast can also be used for production of potable alcohol, including wine, beer, spirits and the like.

Processes for producing alcohols, e.g., ethanol, from carbohydrate substrates are generally well known. The conversion of a carbohydrate substrate to a fermentation product such as alcohol typically involves multiple processing steps and reagents, each of which is important for maximizing conversion rate, efficiency, throughput, consistency, concentration, and/or yield. By way of example, a common production process for producing ethanol by fermentation of a carbohydrate substrate (see, e.g., Section IX below) typically involves processes including, but not limited to, milling or grinding feedstock, liquefaction, saccharification, fermentation, and distillation, and reagents such as enzymes and additional components, e.g., microorganisms and/or chemicals, may be added during a unit operation to facilitate the reaction. For example, during alcohol production, liquefaction includes thinning a mash via partial hydrolysis to lower its viscosity. Enzymes, e.g., alpha-amylases, may be used to facilitate the thinning process. During saccharification, complex carbohydrates of the thinned mash are converted to monosaccharides. Again, enzymes, e.g., glucoamylases, may be used to facilitate the conversion process. The resulting sugars are then converted by yeasts to ethanol during the fermentation process step. It is also possible to include enzymes, e.g., glucoamylases, fungal alpha amylase, and trehalase, to control the glucose profile during fermentation. Reagents, such as enzymes, may also be used in the steps of milling and distillation to facilitate the process. In some cases, the saccharification and fermentation steps may be combined into a single simultaneous saccharification and fermentation (SSF) step. Other well-known processes include raw starch hydrolysis (RSH), which includes milling the starch-containing material to produce a carbohydrate substrate and then performing SSF below the initial gelatinization temperature. Enzymes, e.g., acid fungal amylases, glucoamylases, may also be used in such processes to produce fermentation products.

In some embodiments, the modified yeast described herein are used in a process for producing a fermentation product. Thus, in an aspect is provided a method for producing a fermentation product, including fermenting a carbohydrate substrate with a modified yeast cell described herein. In some embodiments, the method includes a liquefaction step, optionally including an alpha-amylase. In some embodiments, the method includes a saccharification step, optionally including a glucoamylase. In some embodiments, the fermentation product is ethanol and/or butanol. In some embodiments, the fermentation product is ethanol.

As described above, the modified yeast cells described herein are capable of increased ethanol production during fermentation. In some embodiments, the increase in ethanol is at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% more compared to the amount of ethanol produced by parental cells under the same fermentation conditions. In some embodiments, the increase in ethanol is at least about 0.1%, 0.5%, 1%, 2%, 3%, 4%, 5%, 6%, 7%, 8%, 9%, or 10% more compared to the amount of ethanol produced by parental cells under the same fermentation conditions. In some embodiments, the increase in ethanol is at least about 0.1%, 0.5%, 1%, 2%, 3%, 4%, or 5% more compared to the amount of ethanol produced by parental cells under the same fermentation conditions. In some embodiments, the increase in ethanol is in a range of about 0.1% to 15% compared to the amount of ethanol produced by parental cells under the same fermentation conditions. In some embodiments, the increase in ethanol is in a range of about 0.5% to 15% compared to the amount of ethanol produced by parental cells under the same fermentation conditions. In some embodiments, the increase in ethanol is in a range of about 1% to 15% compared to the amount of ethanol produced by parental cells under the same fermentation conditions. In some embodiments, the increase in ethanol is in a range of about 0.1% to 10% compared to the amount of ethanol produced by parental cells under the same fermentation conditions. In some embodiments, the increase in ethanol is in a range of about 0.5% to 10% compared to the amount of ethanol produced by parental cells under the same fermentation conditions. In some embodiments, the increase in ethanol is in a range of about 1% to 10% compared to the amount of ethanol produced by parental cells under the same fermentation conditions. In some embodiments, the increase in ethanol is in a range of about 1% to 5% compared to the amount of ethanol produced by parental cells under the same fermentation conditions. The terms “increased”, “elevated”, “enhanced”, “greater than”, “improved”, “more”, and the like are used interchangeably herein.

In some embodiments, the modified yeast cells described herein produce a decreased amount of acetate compared to the parental cells under the same fermentation conditions. In some embodiments, the decrease in acetate is at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% less compared to the amount of acetate produced by parental cells under the same fermentation conditions. In some embodiments, the decrease in acetate is at least about 0.1%, 0.5%, 1%, 2%, 3%, 4%, 5%, 6%, 7%, 8%, 9%, 10%, 20%, 25%, or 30% less compared to the amount to the amount of acetate produced by parental cells under the same fermentation conditions. In some embodiments, the decrease in acetate is at least about 0.1%, 0.5%, 1%, 2%, 3%, 4%, 5%, 6%, 7%, 8%, 9%, 10%, 20%, or 25% less compared to the amount to the amount of acetate produced by parental cells under the same fermentation conditions. In some embodiments, the decrease in acetate is at least about 0.1%, 0.5%, 1%, 2%, 3%, 4%, 5%, 6%, 7%, 8%, 9%, 10%, or 20% less compared to the amount to the amount of acetate produced by parental cells under the same fermentation conditions. In some embodiments, the decrease in acetate is at least about 0.1%, 0.5%, 1%, 2%, 3%, 4%, 5%, 6%, 7%, 8%, 9%, or 10% less compared to the amount to the amount of acetate produced by parental cells under the same fermentation conditions. In some embodiments, the decrease in acetate is at least about 0.1%, 0.5%, 1%, 2%, 3%, 4%, or 5% less compared to the amount of acetate produced by parental cells under the same fermentation conditions. In some embodiments, the decrease in acetate is in a range of about 0.1% to 30% compared to the amount to of acetate produced by parental cells under the same fermentation conditions. In some embodiments, the decrease in acetate is in a range of about 1% to 30% compared to the amount of acetate produced by parental cells under the same fermentation conditions. In some embodiments, the decrease in acetate is in a range of about 5% to 30% compared to the amount of acetate produced by parental cells under the same fermentation conditions. In some embodiments, the decrease in acetate is in a range of about 10% to 25% compared to the amount of acetate produced by parental cells under the same fermentation conditions. In some embodiments, the decrease in acetate is in a range of about 5% to 10% compared to the amount of acetate produced by parental cells under the same fermentation conditions. In some embodiments, the decrease in acetate is in a range of about 1% to 5% compared to the amount of acetate produced by parental cells under the same fermentation conditions. The terms “decreased,” “lowered,” “reduced,” “less”, “less than,” and the like are used interchangeably herein.

In some embodiments, the modified yeast cells described herein produce a decreased amount of glycerol compared to the parental cells under the same fermentation conditions. In some embodiments, the decrease in glycerol is at least about 0.1%, 0.5%, 1%, 2%, 3%, 4%, 5%, 6%, 7%, 8%, 9%, 10%, 15%, or 20% less compared to the amount of glycerol produced by parental cells under the same fermentation conditions. In some embodiments, the decrease in glycerol is at least about 0.1%, 0.5%, 1%, 2%, 3%, 4%, 5%, 6%, 7%, 8%, 9%, 10%, or 15%, less compared to the amount of glycerol produced by parental cells under the same fermentation conditions. In some embodiments, the decrease in glycerol is at least about 0.1%, 0.5%, 1%, 2%, 3%, 4%, 5%, 6%, 7%, 8%, 9%, or 10% less compared to the amount of glycerol produced by parental cells under the same fermentation conditions. In some embodiments, the decrease in glycerol is at least about 0.1%, 0.5%, 1%, 2%, 3%, 4%, or 5% less compared to the amount of glycerol produced by parental cells under the same fermentation conditions. In some embodiments, the decrease in glycerol is in a range of about 0.1% to 15% compared to the amount to of glycerol produced by parental cells under the same fermentation conditions. In some embodiments, the decrease in glycerol is in a range of about 1% to 15% compared to the amount of glycerol produced by parental cells under the same fermentation conditions. In some embodiments, the decrease in glycerol is in a range of about 1% to 10% compared to the amount of glycerol produced by parental cells under the same fermentation conditions. In some embodiments, the decrease in glycerol is in a range of about 5% to 10% compared to the amount of glycerol produced by parental cells under the same fermentation conditions.

In some embodiments, the modified yeast cells described herein produce a decreased amount of residual glucose compared to the parental cells under the same fermentation conditions. In some embodiments, the decrease in residual glucose is at least about 1%, 2%, 3%, 4%, 5%, 6%, 7%, 8%, 9%, 10%, 15%, 20%, 30%, 40%, 50%, 60%, 70%, or 80% less compared to the amount of residual glucose produced by parental cells under the same fermentation conditions. In some embodiments, the decrease in residual glucose is at least about 1%, 2%, 3%, 4%, 5%, 6%, 7%, 8%, 9%, 10%, 15%, 20%, 30%, 40%, 50%, 60%, or 70% less compared to the amount of residual glucose produced by parental cells under the same fermentation conditions. In some embodiments, the decrease in residual glucose is at least about 1%, 2%, 3%, 4%, 5%, 6%, 7%, 8%, 9%, 10%, 15%, 20%, 30%, 40%, 50%, or 60% less compared to the amount of residual glucose produced by parental cells under the same fermentation conditions. In some embodiments, the decrease in residual glucose is in a range of about 1% to 70% compared to the amount to of residual glucose produced by parental cells under the same fermentation conditions. In some embodiments, the decrease in residual glucose is in a range of about 10% to 70% compared to the amount to of residual glucose produced by parental cells under the same fermentation conditions. In some embodiments, the decrease in residual glucose is in a range of about 20% to 70% compared to the amount to of residual glucose produced by parental cells under the same fermentation conditions. In some embodiments, the decrease in residual glucose is in a range of about 30% to 70% compared to the amount of residual glucose produced by parental cells under the same fermentation conditions. In some embodiments, the decrease in residual glucose is in a range of about 40% to 70% compared to the amount of residual glucose produced by parental cells under the same fermentation conditions. In some embodiments, the decrease in residual glucose is in a range of about 50% to 65% compared to the amount to of residual glucose produced by parental cells under the same fermentation conditions.

In some embodiments, the modified yeast cells described herein produce ethanol at an increased rate compared to the rate of ethanol produced by parental cells under the same fermentation conditions. In some embodiments, the increase in rate is at least about 0.1%, 0.5%, 1%, 2%, 3%, 4%, 5%, 6%, 7%, 8%, 9%, or 10% faster compared to the rate of ethanol produced by parental cells under the same fermentation conditions. In some embodiments, the increased rate occurs early in the fermentation conditions, e.g., between about 0 to about 22 hrs in the fermentation condition.

In some embodiments, the modified yeast described herein produce an increased amount of ethanol and a decreased amount of glycerol as described herein. In some embodiments, the modified yeast described herein produce an increased amount of ethanol and a decreased amount of acetate as described herein. In some embodiments, the modified yeast described herein produce an increased amount of ethanol and a decreased amount of glycerol and a decreased amount of acetate as described herein. In some embodiments, the modified yeast described herein produce a decreased amount of glycerol and a decreased amount of acetate as described herein. In some embodiments, the modified yeast described herein produce ethanol at a faster rate as described herein.

### VIII. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Pichia, Issatchenkia, Kluyveromyces, Lachancea and Schizosaccharomyces spp. In some embodiments, the yeast is S. cerevisiae.

Numerous yeast strains are commercially available, many of which have been selected or genetically engineered for desired characteristics, such as high alcohol production, rapid growth rate. In some embodiments, the yeast is already genetically modified, whether using standard molecular biology techniques, by chemical mutagenesis, by mating, by stress-selection and adaptation or by other methods, to improve ethanol production in fuel ethanol plants.

### IX. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present modified yeast cells and methods are believed to be fully compatible with such substrates and conditions.

Alcohol fermentation products include organic compounds having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present yeast strains and methods will be apparent to the skilled person in view of the present description. The following examples are intended to further illustrate, but not limit, the modified yeast cells and methods.

### EXAMPLES

### Example 1

### Materials and Methods

Liquefact preparation: Liquefact (corn mash slurry) was prepared by adding 600 ppm of urea, 0.124 SAPU/g ds acid fungal protease, 0.33 GAU/g ds variant Trichoderma reesei glucoamylase and 1.46 SSCU/g ds Aspergillus kawachii α-amylase, adjusted to a pH of 4.8 with sulfuric acid.

ANKOM assays:

300 μL of concentrated yeast overnight culture was added to each of a number ANKOM bottles filled with 50 g prepared liquefact (see above) to a final OD of 0.3. The bottles were then incubated at 32° C. with shaking at 150 RPM for 55 hours.

HPLC analysis:

Samples of the cultures from ANKOM assays were collected in Eppendorf tubes by centrifugation for 12 minutes at 14,000 RPM. The supernatants were filtered using 0.2 μM PTFE filters and then used for HPLC (Agilent Technologies 1200 series) analysis with the following conditions: Bio-Rad Aminex HPX-87H columns, running temperature of 55° C. 0.6 ml/min isocratic flow 0.01 N H₂SO₄, 2.5 μl injection volume. Calibration standards were used for quantification of the acetate, ethanol, glycerol, glucose and other molecules. All values are reported in g/L.

### Example 2

Generation of Yeast Strains Expressing RuBisCo from G. Capsiferriformans

Strain G5308 harbors the gene encoding the RuBisCo gene from proteobacteria Thiobacillus denitrificans, along with genes encoding Escherichia coli chaperones GroEL (SEQ ID NOs: 3 and 4) and GroES (SEQ ID NOs: 5 and 6), Spinacia oleracea phosphoribulose kinase (PRK1; SEQ ID NOs: 9 and 10), and a glycerol reuptake cassette consisting of the glycerol transporter STL1 from Zygosaccharomyces rouxii (SEQ ID NOs: 13 and 14), dihydroxyacetone kinase DAK1 from Saccharomyces cerevisiae (SEQ ID NOs: 11 and 12), and glycerol dehydrogenase GldA from E. coli (SEQ ID NOs: 7 and 8).

Using CRISPR-mediated integration, a gene encoding the CbbM/RuBisCo enzyme from Gallionella capsiferriformans (GenBank Accession No. CP002159; herein “GcRuBisCo”) was introduced into the parental yeast strain at the site of the T. denitrificans CbbM gene. Briefly, a guide RNA was designed that was specific to the existing T. denitrificans CbbM, which was then was replaced in situ by a codon-optimized version of GcRuBisCo for functional expression in S. cerevisiae using the existing S. cerevisiae TDH3 promoter (GenBank CP020129) and S. cerevisiae CTC1 terminator (GenBank KM407505.1) remaining in the genome from the T. denitrificans CbbM gene.

is shown, below, as SEQ ID NO: 1:
MDQSNRYANLNLKEEDLIKNGKHLLVAYKLIPAKGHGFLEVAAHVAAESS
TGTNVEVSTTDDFTRGVDALVYEIDETAFGDDIVKGGGLFKVAYPVELFD
PNLTDGTYNISHMWSLILGNNQGMGDHQGLRMLDFLVPEMMVRKFDGPSA
NISNLWKVLGRSETDGGYIAGTIIKPKLGLRPEPFAKACYDFWLGGDFIK
NDEPQANQPFCPMEVVMPKVAEAMDRAQQETGQAKLFSANITADYYKEMI
HRGDFVLETFAKYNSASHVAFLVDGFVTGPAGVTTCRREFPDTFLHFHRA
GHGAVTSYKSPMGMDPLCYMKLVRLMGASGMHTGTMGYGKMEGHGKETVL
AYMLERDECQGPYFYQKWYGMKATTPIISGGMNALRLPGFFQNLGHGNVI
NTCGGGAFGHIDSPAAGGISLGQAYDCWKSGSDPIEYAKTHKEFARAFES
FPKDGDKLFAGWREKLGVHK
The optimized coding region of the nucleic
acid sequence of the gene encoding the
GcRuBisCo is shown, below, as SEQ ID NO: 2:
ATGGACCAATCCAACAGATACGCCAATTTGAACTTGAAGGAAGAAGACTT
GATCAAGAACGGTAAGCACTTATTGGTTGCTTACAAGCTAATTCCAGCCA
AAGGTCACGGCTTCTTGGAAGTCGCTGCCCACGTTGCTGCCGAATCTTCC
ACTGGTACCAACGTCGAAGTTTCTACTACCGACGATTTCACCAGAGGTGT
CGACGCTTTGGTTTACGAAATCGACGAAACTGCTTTTGGTGACGATATCG
TCAAGGGTGGTGGCTTGTTCAAGGTTGCTTACCCAGTCGAATTGTTCGAC
CCAAACTTGACCGATGGTACTTACAACATTTCTCACATGTGGTCCTTGAT
CTTAGGCAACAATCAAGGTATGGGCGATCATCAAGGTTTGCGTATGTTGG
ACTTCTTGGTTCCAGAAATGATGGTCAGAAAGTTTGATGGTCCTTCTGCC
AACATTTCCAATTTATGGAAGGTCTTGGGTAGATCCGAAACAGACGGTGG
CTACATTGCTGGTACCATTATCAAACCAAAGTTGGGTTTACGTCCAGAAC
CTTTTGCTAAGGCCTGCTACGACTTCTGGTTAGGTGGCGACTTCATCAAG
AACGACGAACCTCAAGCTAACCAACCATTTTGTCCAATGGAAGTCGTTAT
GCCAAAGGTTGCTGAAGCTATGGACAGAGCTCAACAGGAAACTGGTCAAG
CCAAGTTGTTCTCTGCTAACATTACTGCTGACTATTACAAGGAAATGATT
CACAGAGGTGACTTTGTTCTAGAAACTTTTGCCAAGTACAACTCTGCTTC
CCACGTTGCTTTCTTGGTCGATGGTTTCGTTACTGGTCCAGCTGGTGTTA
CCACTTGTAGACGTGAATTTCCAGATACCTTCTTGCACTTTCACAGAGCT
GGTCACGGTGCTGTTACCTCCTACAAGTCTCCAATGGGTATGGATCCATT
GTGTTACATGAAGTTGGTCAGATTGATGGGTGCTTCTGGTATGCATACCG
GTACTATGGGTTACGGCAAGATGGAAGGTCACGGCAAGGAAACCGTCTTG
GCTTACATGTTGGAAAGAGACGAATGTCAAGGTCCATACTTCTACCAAAA
GTGGTACGGTATGAAGGCTACCACTCCAATCATTTCTGGTGGCATGAACG
CTTTGAGACTACCAGGTTTCTTTCAAAACTTGGGTCACGGCAACGTCATC
AATACTTGTGGTGGCGGTGCCTTTGGTCACATCGACTCTCCAGCTGCCGG
TGGCATCTCCTTGGGTCAAGCTTACGACTGTTGGAAGTCTGGTTCCGATC
CAATCGAATACGCCAAGACCCACAAGGAATTCGCTAGAGCCTTCGAATCC
TTTCCAAAGGATGGCGACAAGTTGTTCGCTGGCTGGAGAGAAAAGTTGGG
TGTTCACAAGTAA

When cultivated in a SSF process, yeast cells expressing GcRuBisCo (G5529) showed a significant reduction in glycerol and acetate combined with no change (or even a moderate increase) in ethanol production (Table 1).

TABLE 1
End of fermentation analysis of yeast
expressing different RuBisCo enzymes

| Strain | RuBisCo source | Ethanol | Glucose | Glycerol | Acetate |
|---|---|---|---|---|---|
| G5308 | T. denitrificans | 151.2 | 2.71 | 8.23 | 0.49 |
| G5529 | G. capsiferriformans | 152.2 | 0.98 | 7.46 | 0.40 |

Glycerol production was reduced by 9.26% and acetate was reduced by 19.47%, following fermentation with yeast cells expressing the GcRuBisCo compared to following fermentation with yeast cells expressing the RuBisCo enzyme from T. denitrificans. Residual glucose was also decreased by over 60%.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. Modified yeast cells derived from parental yeast cells, the modified yeast cells comprising a genetic alteration that causes the modified cells to produce a ribulose-1,5-bisphosphate carboxylase-oxygenase (RuBisCo) enzyme from Gallionella capsiferriformans (GcRuBisCo).

2. The modified yeast cells of claim 1, further comprising a gene encoding a phosphoribulokinase.

3. Modified yeast cells derived from parental yeast cells, the modified cells comprising a genetic alteration that causes the modified cells to produce the ribulose-1,5-bisphosphate carboxylase-oxygenase (RuBisCo) enzyme from Gallionella capsiferriformans (GcRuBisCo), wherein the modified cells produce during fermentation a decreased amount of glycerol and/or acetate compared to the amount of glycerol and acetate produced by otherwise identical parental cells producing the RuBisCo enzyme from Thiobacillus dentrificans under identical fermentation conditions, and wherein the modified yeast cells and parental yeast cells both further comprise an exogenous gene encoding a phosphoribulokinase.

4. The modified yeast cells of any one of claims 1-3, wherein the genetic alteration comprises introduction into the parental cells a nucleic acid capable of directing the expression of GcRuBisCo.

5. The modified yeast cells of any one of claims 1-4, wherein the genetic alteration comprises introduction of an expression cassette for directing the expression of the GcRuBisCo.

6. The modified yeast cells of any one of claims 1-5, wherein the GcRuBisCo comprises an amino acid sequence set forth by SEQ ID NO: 1 or an amino acid sequence having at least 70% identity to the sequence set forth by SEQ ID NO: 1.

7. The modified yeast cells of any one of claims 1-6, further comprising exogenous genes encoding chaperone proteins.

8. The modified yeast cells of claim 7, where the chaperone proteins are GroES and GroEL from Escherichia coli.

9. The modified yeast cells of claim 8, wherein the GroES comprises an amino acid sequence set forth by SEQ ID NO: 5, or an amino acid sequence having at least 70% identity to SEQ ID NO: 5; and/or
the GroEL comprises an amino acid sequence set forth by SEQ ID NO: 3, or an amino acid sequence having at least 70% identity to SEQ ID NO: 3.

10. The modified yeast cells of any one of claims 1-9, further comprising an exogenous gene encoding a glycerol transporter, a glycerol dehydrogenase and dihydroxyacetone kinase.

11. The modified yeast cells of any one of claims 1-10, wherein the cells further comprise one or more genes of the phosphoketolase pathway.

12. The modified yeast cells of any one of claims 1-11, further comprising an alteration in the glycerol pathway and/or the acetyl-CoA pathway.

13. The modified yeast cells of any one of claims 1-12, further comprising an alternative pathway for making ethanol.

14. The modified yeast cells of any one of claims 2-13, wherein the phosphoribulokinase comprises an amino acid sequence set forth by SEQ ID NO: 9, or an amino acid sequence having at least 70% identity to SEQ ID NO: 9.

15. The modified yeast cells of any one of claims 1-14, wherein the cells further comprise an exogenous gene encoding a carbohydrate processing enzyme.

16. The modified yeast cells of any of claims 1-15, wherein the cells are of a Saccharomyces spp.

17. A method for decreasing the production of glycerol and acetate from yeast cells grown on a carbohydrate substrate, comprising: introducing into parental yeast cells a genetic alteration that causes the modified yeast cells to produce ribulose-1,5-bisphosphate carboxylase-oxygenase (RuBisCo) from Gallionella capsiferriformans.

18. The method of claim 17, wherein the modified yeast cells are the modified yeast cells of any of claims 1-16.

19. A method for producing a fermentation product, comprising fermenting a carbohydrate substrate with a modified yeast cell of any one of claims 1-16 under conditions for producing a fermentation product.

20. The method of claim 20, wherein the fermentation product is ethanol.


SEQUENCE LISTING (14 sequences)

SEQ ID NO: 1 - protein, 470 aa, Gallionella capsiferriformans
MDQSNRYANLNLKEEDLIKNGKHLLVAYKLIPAKGHGFLEVAAHVAAESSTGTNVEVSTTDDFTRGVDALVYEIDETAFGDDIVKGGGLFKVAYPVELFDPNLTDGTYNISHMWSLILGNNQGMGDHQGLRMLDFLVPEMMVRKFDGPSANISNLWKVLGRSETDGGYIAGTIIKPKLGLRPEPFAKACYDFWLGGDFIKNDEPQANQPFCPMEVVMPKVAEAMDRAQQETGQAKLFSANITADYYKEMIHRGDFVLETFAKYNSASHVAFLVDGFVTGPAGVTTCRREFPDTFLHFHRAGHGAVTSYKSPMGMDPLCYMKLVRLMGASGMHTGTMGYGKMEGHGKETVLAYMLERDECQGPYFYQKWYGMKATTPIISGGMNALRLPGFFQNLGHGNVINTCGGGAFGHIDSPAAGGISLGQAYDCWKSGSDPIEYAKTHKEFARAFESFPKDGDKLFAGWREKLGVHK

SEQ ID NO: 2 - DNA, 1413 bp, Synthetic construct
  source 1..1413: mol_type = other DNA
atggaccaatccaacagatacgccaatttgaacttgaaggaagaagacttgatcaagaacggtaagcacttattggttgcttacaagctaattccagccaaaggtcacggcttcttggaagtcgctgcccacgttgctgccgaatcttccactggtaccaacgtcgaagtttctactaccgacgatttcaccagaggtgtcgacgctttggtttacgaaatcgacgaaactgcttttggtgacgatatcgtcaagggtggtggcttgttcaaggttgcttacccagtcgaattgttcgacccaaacttgaccgatggtacttacaacatttctcacatgtggtccttgatcttaggcaacaatcaaggtatgggcgatcatcaaggtttgcgtatgttggacttcttggttccagaaatgatggtcagaaagtttgatggtccttctgccaacatttccaatttatggaaggtcttgggtagatccgaaacagacggtggctacattgctggtaccattatcaaaccaaagttgggtttacgtccagaaccttttgctaaggcctgctacgacttctggttaggtggcgacttcatcaagaacgacgaacctcaagctaaccaaccattttgtccaatggaagtcgttatgccaaaggttgctgaagctatggacagagctcaacaggaaactggtcaagccaagttgttctctgctaacattactgctgactattacaaggaaatgattcacagaggtgactttgttctagaaacttttgccaagtacaactctgcttcccacgttgctttcttggtcgatggtttcgttactggtccagctggtgttaccacttgtagacgtgaatttccagataccttcttgcactttcacagagctggtcacggtgctgttacctcctacaagtctccaatgggtatggatccattgtgttacatgaagttggtcagattgatgggtgcttctggtatgcataccggtactatgggttacggcaagatggaaggtcacggcaaggaaaccgtcttggcttacatgttggaaagagacgaatgtcaaggtccatacttctaccaaaagtggtacggtatgaaggctaccactccaatcatttctggtggcatgaacgctttgagactaccaggtttctttcaaaacttgggtcacggcaacgtcatcaatacttgtggtggcggtgcctttggtcacatcgactctccagctgccggtggcatctccttgggtcaagcttacgactgttggaagtctggttccgatccaatcgaatacgccaagacccacaaggaattcgctagagccttcgaatcctttccaaaggatggcgacaagttgttcgctggctggagagaaaagttgggtgttcacaagtaa

SEQ ID NO: 3 - protein, 548 aa, Escherichia coli
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMGGMGGMM

SEQ ID NO: 4 - DNA, 1647 bp, Synthetic construct
  source 1..1647: mol_type = other DNA
atggctgctaaggacgttaagttcggtaacgacgctagagttaagatgttgagaggtgttaacgttttggctgacgctgttaaggttactttgggtccaaagggtagaaacgttgttttggacaagtctttcggtgctccaactatcactaaggacggtgtttctgttgctagagaaatcgaattggaagacaagttcgaaaacatgggtgctcaaatggttaaggaagttgcttctaaggctaacgacgctgctggtgacggtactactactgctactgttttggctcaagctatcatcactgaaggtttgaaggctgttgctgctggtatgaacccaatggacttgaagagaggtatcgacaaggctgttactgctgctgttgaagaattgaaggctttgtctgttccatgttctgactctaaggctatcgctcaagttggtactatctctgctaactctgacgaaactgttggtaagttgatcgctgaagctatggacaaggttggtaaggaaggtgttatcactgttgaagacggtactggtttgcaagacgaattggacgttgttgaaggtatgcaattcgacagaggttacttgtctccatacttcatcaacaagccagaaactggtgctgttgaattggaatctccattcatcttgttggctgacaagaagatctctaacatcagagaaatgttgccagttttggaagctgttgctaaggctggtaagccattgttgatcatcgctgaagacgttgaaggtgaagctttggctactttggttgttaacactatgagaggtatcgttaaggttgctgctgttaaggctccaggtttcggtgacagaagaaaggctatgttgcaagacatcgctactttgactggtggtactgttatctctgaagaaatcggtatggaattggaaaaggctactttggaagacttgggtcaagctaagagagttgttatcaacaaggacactactactatcatcgacggtgttggtgaagaagctgctatccaaggtagagttgctcaaatcagacaacaaatcgaagaagctacttctgactacgacagagaaaagttgcaagaaagagttgctaagttggctggtggtgttgctgttatcaaggttggtgctgctactgaagttgaaatgaaggaaaagaaggctagagttgaagacgctttgcacgctactagagctgctgttgaagaaggtgttgttgctggtggtggtgttgctttgatcagagttgcttctaagttggctgacttgagaggtcaaaacgaagaccaaaacgttggtatcaaggttgctttgagagctatggaagctccattgagacaaatcgttttgaactgtggtgaagaaccatctgttgttgctaacactgttaagggtggtgacggtaactacggttacaacgctgctactgaagaatacggtaacatgatcgacatgggtatcttggacccaactaaggttactagatctgctttgcaatacgctgcttctgttgctggtttgatgatcactactgaatgtatggttactgacttgccaaagaacgacgctgctgacttgggtgctgctggtggtatgggtggtatgggtggtatgggtggtatgatgtaa

SEQ ID NO: 5 - protein, 548 aa, Escherichia coli; identical to SEQ ID NO: 3

SEQ ID NO: 6 - DNA, 294 bp, Synthetic construct
  source 1..294: mol_type = other DNA
atgaacatcagaccattgcacgacagagttatcgttaagagaaaggaagttgaaactaagtctgctggtggtatcgttttgactggttctgctgctgctaagtctactagaggtgaagttttggctgttggtaacggtagaatcttggaaaacggtgaagttaagccattggacgttaaggttggtgacatcgttatcttcaacgacggttacggtgttaagtctgaaaagatcgacaacgaagaagttttgatcatgtctgaatctgacatcttggctatcgttgaagcttaa

SEQ ID NO: 7 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 8 - DNA, 1104 bp, Synthetic construct
  source 1..1104: mol_type = other DNA
atggacagaatcatccaatctccaggtaagtacatccaaggtgctgatgttatcaacagattaggtgaatacttgaagccattggctgaaagatggttagtcgtcggtgacaaattcgttttgggtttcgctcaatccaccgtcgaaaagtctttcaaggatgctggtttggttgttgaaatcgctccattcggtggtgaatgttctcaaaatgaaattgaccgtttgagaggtattgctgaaactgctcaatgtggtgccatcttgggtattggtggtggtaagactttggacactgccaaggctttggcccacttcatgggtgttccagttgccattgctccaaccattgcttctaccgatgctccatgttctgctttgtccgttatctacaccgacgaaggtgaatttgaccgttacttgttgttgccaaacaacccaaacatggtcattgtcgacaccaagatcgttgccggtgctccagccagattattggctgccggtatcggtgatgctttggctacctggttcgaagccagagcttgttccagatctggtgctactaccatggccggtggtaaatgtactcaagctgctttagctttggctgaattgtgttacaacactttgttggaagaaggcgaaaaggctatgttggctgctgaacaacacgttgttactccagctttggaaagagtcattgaagccaacacctacttgtccggtgttggattcgaatctggtggtttagctgccgctcatgccgtccacaacggtttgactgccatcccagatgctcaccactactaccacggtgaaaaggttgctttcggtactttgactcaattagtcttggaaaacgctccagtcgaagaaatcgaaaccgttgctgctctatcccacgctgtcggtttgcctatcactttggctcaattggacatcaaggaagatgtcccagctaagatgagaattgttgctgaagctgcttgtgctgaaggtgaaaccattcacaacatgccaggtggtgccaccccagaccaagtctacgctgctttgttggttgctgaccaatacggtcaaagattcttgcaagaatgggagtaa

SEQ ID NO: 9 - protein, 352 aa, Spinacea oleracea
MSQQQTIVIGLAADSGCGKSTFMRRLTSVFGGAAEPPKGGNPDSNTLISDTTTVICLDDFHSLDRNGRKVEKVTALDPKANDFDLMYEQVKALKEGKAVDKPIYNHVSGLLDPPELIQPPKILVIEGLHPMYDARVRELLDFSIYLDISNEVKFAWKIQRDMKERGHSLESIKASIESRKPDFDAYIDPQKQHADVVIEVLPTELIPDDDEGKVLRVRMIQKEGVKFFNPVYLFDEGSTISWIPCGRKLTCSYPGIKFSYGPDTFYGNEVTVVEMDGMFDRLDELIYVESHLSNLSTKFYGEVTQQMLKHQNFPGSNNGTGFFQTIIGLKIRDLFEQLVASRSTATATAAKA

SEQ ID NO: 10 - DNA, 1059 bp, Synthetic construct
  source 1..1059: mol_type = other DNA
atgtcacaacaacaaacaattgtgattggtttagcagcagattcaggttgtggtaagagtacattcatgaggaggttaacaagtgttttcggtggcgcggccgagccaccaaagggtggtaacccagattcaaacacattgattagtgacactactactgttatctgtttggatgattttcattcccttgatagaaatggcaggaaagtggaaaaagttactgctttagacccaaaagctaatgattttgatcttatgtatgaacaagttaaggctttgaaagaaggtaaagctgttgataaacctatttataatcatgtttctggtttgttggaccctcctgagcttattcaacctcctaagatcttggtcattgaagggttacaccccatgtatgacgcacgtgtgagggaattgctagacttcagcatctacttggacattagcaatgaagttaaatttgcctggaaaattcagagagacatgaaagaaagaggacacagtcttgaaagcatcaaagccagtattgaatccagaaagccagattttgatgcttacattgacccacaaaagcagcatgctgatgtagtgattgaagtattgccaactgaactcattcctgatgatgatgaaggcaaagtgttgagagtaaggatgattcagaaagaaggagtcaagtttttcaacccagtttacttgtttgatgaaggatctaccatttcatggattccatgtggtagaaaattaacgtgttcttaccctggtatcaaattttcctatggcccagacaccttctatggcaacgaggtgacagtagtagagatggatggaatgtttgacagattagacgaactaatctacgtcgaaagccatttgagcaatctatcaaccaagttttatggtgaagtcactcaacaaatgttgaagcaccaaaatttcccaggaagcaacaatggaactggtttcttccaaaccataattggattgaagatcagagacttgttcgagcagctcgttgctagcaggtctacagcaactgcaacagctgctaaagcctaa

SEQ ID NO: 11 - protein, 584 aa, Saccharomyces cerevisiae
MSAKSFEVTDPVNSSLKGFALANPSITLVPEEKILFRKTDSDKIALISGGGSGHEPTHAGFIGKGMLSGAVVGEIFASPSTKQILNAIRLVNENASGVLLIVKNYTGDVLHFGLSAERARALGINCRVAVIGDDVAVGREKGGMVGRRALAGTVLVHKIVGAFAEEYSSKYGLDGTAKVAKIINDNLVTIGSSLDHCKVPGRKFESELNEKQMELGMGIHNEPGVKVLDPIPSTEDLISKYMLPKLLDPNDKDRAFVKFDEDDEVVLLVNNLGGVSNFVISSITSKTTDFLKENYNITPVQTIAGTLMTSFNGNGFSITLLNATKATKALQSDFEEIKSVLDLLNAFTNAPGWPIADFEKTSAPSVNDDLLHNEVTAKAVGTYDFDKFAEWMKSGAEQVIKSEPHITELDNQVGDGDCGYTLVAGVKGITENLDKLSKDSLSQAVAQISDFIEGSMGGTSGGLYSILLSGFSHGLIQVCKSKDEPVTKEIVAKSLGIALDTLYKYTKARKGSSTMIDALEPFVKEFTASKDFNKAVKAAEEGAKSTATFEAKFGRASYVGDSSQVEDPGAVGLCEFLKGVQSAL

SEQ ID NO: 12 - DNA, 1755 bp, Synthetic construct
  source 1..1755: mol_type = other DNA
atgtccgctaaatctttcgaagttaccgacccagtcaactcttctttgaagggttttgctttggccaacccatccattactttggtcccagaagaaaagatcttattcagaaagactgactctgacaaaattgctttgatctccggtggtggttccggtcacgaaccaacccacgctggtttcatcggtaagggtatgttgtccggtgctgtcgttggtgaaatctttgcttctccatccaccaagcaaatcttgaatgctatcagattagtcaacgaaaacgcttctggtgtcttgttgattgtcaagaactacactggtgacgtcttgcatttcggtttatctgctgaaagagctagagctttgggtattaactgtagagttgccgtcatcggtgacgatgttgctgtcggtcgtgaaaagggtggtatggttggtagacgtgctttggctggtactgtcttggttcacaagattgttggtgctttcgctgaagaatactcctccaagtacggtttagatggtactgctaaggttgccaagatcatcaacgacaacttggttaccatcggttcttctttggaccactgtaaggttccaggtagaaagttcgaatctgaattgaacgaaaagcaaatggaattgggtatgggtatccacaacgaaccaggtgttaaggtcttggacccaattccatccactgaagatttgatttccaaatacatgttgccaaagttgctagacccaaacgacaaggacagagctttcgttaagttcgatgaagatgacgaagttgttttgttggtcaacaacttgggtggtgtttctaacttcgtcatctcttctattacctccaagaccaccgatttcttaaaggaaaactacaacatcactccagtccaaaccattgccggtactttgatgacctctttcaacggtaacggtttctccatcaccttgttgaatgccaccaaagctaccaaggctttgcaatctgatttcgaagaaatcaaatccgtcttagatttgttgaacgccttcaccaacgccccaggttggccaattgctgacttcgaaaagacctctgctccatctgttaacgatgacttgttgcacaacgaagttactgccaaggccgtcggtacttacgatttcgacaaattcgctgaatggatgaagtctggtgctgaacaagtcatcaaatctgaaccacacatcactgaattggacaaccaagttggtgatggtgactgtggttacactttggttgctggtgtcaagggtatcactgaaaacttggacaaattgtccaaggactctttgtctcaagctgttgctcaaatttctgatttcattgaaggttccatgggtggtacttctggtggtttgtactccatcttgttgtctggtttctcccacggtttgatccaagtttgtaagtccaaggatgaacctgtcaccaaggaaattgttgccaagtctctaggtattgctttggacactttatacaagtacaccaaggccagaaagggttcttccaccatgatcgatgctttggaaccatttgtcaaggaattcactgcttctaaggacttcaacaaggctgttaaggctgctgaagaaggtgccaagtccactgctactttcgaagctaagttcggtagagcttcttacgttggtgactcttctcaagttgaagatccaggtgctgttggtttatgtgaattcttgaagggtgtccaatctgcgctttaa

SEQ ID NO: 13 - protein, 592 aa, Zygosaccharomyces rouxii
MGKRTQGFMDYVFSRTSTAGLKGARLRYTAAAVAVIGFALFGYDQGLMSGLITGDQFNKEFPPTKSNGDNDRYASVIQGAVTACYEIGCFFGSLFVLFFGDAIGRKPLIIFGAIIVIIGTVISTAPFHHAWGLGQFVVGRVITGVGTGFNTSTIPVWQSEMTKPNIRGAMINLDGSVIAFGTMIAYWLDFGFSFINSSVQWRFPVSVQIIFALVLLFGIVRMPESPRWLMAKKRPAEARYVLACLNDLPENDDAILAEMTSLHEAVNRSSNQKSQMKSLFSMGKQQNFSRALIASSTQFFQQFTGCNAAIYYSTVLFQTTVQLDRLLAMILGGVFATVYTLSTLPSFYLVEKVGRRKMFFFGALGQGISFIITFACLVNPTKQNAKGAAVGLYLFIICFGLAILELPWIYPPEIASMRVRAATNAMSTCTNWVTNFAVVMFTPVFIQTSQWGCYLFFAVMNFIYLPVIFFFYPETAGRSLEEIDIIFAKAHVDGTLPWMVAHRLPKLSMTEVEDYSQSLGLHDDENEKEEYDEKEAEANAALFQVETSSKSPSSNRKDDDAPIEHNEVQESNDNSSNSSNVEAPIPVHHNDP

SEQ ID NO: 14 - DNA, 1779 bp, Synthetic construct
  source 1..1779: mol_type = other DNA
atgggtaagagaactcaaggtttcatggactacgttttctccagaacttctactgctggtttgaaaggtgccagattgagatacactgctgctgccgttgctgttatcggtttcgctttattcggttacgaccaaggtttgatgtctggtttgattaccggtgaccaattcaacaaggaattccctccaaccaaatctaacggtgacaacgacagatacgcctccgtcatccaaggtgctgtcactgcttgttacgaaattggttgtttcttcggttctttgttcgtcttattctttggtgatgccatcggtagaaagccattaatcattttcggtgccatcatcgtcatcattggtactgttatctccactgctccattccaccacgcttggggtttgggtcaatttgttgtcggtagagttatcactggtgtcggtactggtttcaacacttccaccatcccagtctggcaatctgaaatgaccaagccaaacatcagaggtgctatgattaacttggatggttccgtcattgctttcggtactatgattgcttactggttggatttcggtttctctttcatcaactcttctgttcaatggagattcccagtttccgttcaaatcatctttgctttggtcttattgttcggtattgtcagaatgccagaatctccaagatggttgatggccaagaagcgtccagctgaagccagatacgttttggcttgtttgaatgacttgccagaaaacgacgatgccatcttggccgaaatgacttctttgcacgaagctgtcaacagatcttctaaccaaaagtctcaaatgaaatctttgttctccatgggtaagcaacaaaacttctccagagctttgattgcttcctctacccaattcttccaacaattcaccggttgtaatgctgccatctactactccactgttttgttccaaaccaccgttcaattagatagattgttggctatgatcttgggtggtgtctttgctaccgtttacaccttgtccactttgccatctttctacttggttgaaaaggttggtcgtcgtaagatgttcttcttcggtgctttaggtcaaggtatttctttcattattacctttgcttgtttggtcaacccaaccaagcaaaacgctaagggtgctgctgtcggtttatacttgttcatcatctgtttcggtttggctatcttggaattgccatggatctacccaccagaaattgcttccatgagagtcagagctgctaccaacgctatgtccacctgtaccaactgggttaccaactttgccgttgtcatgttcaccccagtctttatccaaacttctcaatggggttgttacttgttcttcgctgtcatgaacttcatctacttgcctgttatcttcttcttctacccagaaactgccggtcgttctttggaagaaatcgatattattttcgctaaggctcatgttgacggtactctaccatggatggttgctcacagattaccaaagttgtccatgactgaagtcgaagattactctcaatctttgggtttgcacgacgatgaaaacgaaaaggaagaatacgacgaaaaagaagctgaagctaacgctgccttgttccaagttgaaacctcttccaagtctccatcctccaacagaaaggatgacgacgctccaattgaacacaacgaagttcaagaatctaacgacaactcctctaactcttctaacgttgaagctccaattccagttcaccacaacgacccataa